(19) United States
(12) Patent Application Publication
(10) Pub. No.: US 2025/0285754 A1
(43) Pub. Date: Sep. 11, 2025

(54) ANOMALY DETECTION BASED ON COMPLETE BLOOD COUNTS USING MACHINE LEARING

(71) Applicant: Cambridge Enterprise Limited, Cambridge, Cambridgeshire (GB)

(72) Inventors: Nicholas Stephen Gleadall, Cambridgeshire (GB); Michael Thomas Roberts, Cambridgeshire (GB)

(21) Appl. No.: 18/575,764

(22) PCT Filed: Jul. 1, 2022
(86) PCT No.: PCT/GB2022/051710

§ 371 Date: Dec. 29, 2023

(30) Foreign Application Priority Data
Jul. 1, 2021 (GB) 2109560.9

(51) Int. Cl. G16H 50/20; A61B 5/00; G16H 10/40; G16H 50/50; G16H 50/70; G16H 50/80

(52) U.S. Cl. CPC G16H 50/20; A61B 5/7267; G16H 10/40; G16H 50/50; G16H 50/70; G16H 50/80

(57) ABSTRACT

Herein disclosed is a method of preparing a model to detect health and ill-health related characteristics in complete blood counts (CBC) data. The method comprises receiving CBC data from one or more data sources, where the CBC data comprise raw and rich data; encoding CBC data using one or more machine-learning algorithms; training classifier for biological traits based on the encoded CBC data, where the biological traits comprise disease phenotypes; and outputting the model comprising the trained classifier.

### CROSS REFERENCE TO RELATED APPLICATIONS

This application is a U.S. a national stage application under 35 U.S.C. 371 of PCT/GB2022/051710 (“the '710 PCT application”), filed Jul. 1, 2022, which claims priority to German patent application GB2109560.9, filed Jul. 1, 2021 (“the '560.9 Priority Application”). The contents of the '710 PCT Application and '560.9 Priority application are incorporated herein in their entireties.

### TECHNICAL FIELD

The present application relates to a system, platform, and methods for anomaly detection based on blood counts data using machine learning.

### BACKGROUND

Laboratories, at hospitals, primary care centres, health clinics, amongst others routinely administer complete blood count tests for patients and healthy individuals for detection of disease, monitoring side effects of administered drugs, and assessment of general health amongst many other indications for the test. Members of clinical care teams, including but not limited to clinicians, nurses, midwifes, and health practitioners use the test results to screen widely for disease, transition from health to ill-health, to monitor side effects of drugs, to determine the limits of cancer therapy dosing or assign a precise diagnosis if it concerns an acquired or inherited disease of the blood and immune system. Data collected from the complete blood count tests are used to produce summary test results which are generated by the application of instrument manufacturer algorithms. After the summary data have been reported to the clinical care team, all other rich measurement data are generally discarded. The current usage of the blood counts data is thereby inefficient. The test results often do not paint a complete picture of the health status of the individual from whom the sample of blood has been taken.

There is a need for better utilization of complete blood count data. To address this need, herein describes at least one method, system, platform, medium and/or apparatus to detect anomalous health results based on complete blood count measurement data using machine learning.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides a system, apparatus, and method(s) for anomaly detection based on blood count data using machine learning. The disclosure provides a way to utilize data collected from the complete blood count tests to generate a simulation or method that can be used to detect anomalies in blood count results from individuals or at a population level. The method may be deployed with or on a software platform, comprising one or more hardware devices configured to pre-process the cell blood count data. The data generated from the model may be reported to the clinical care team for more efficient utilization.

In a first aspect, the present disclosure provides a method or computer-implemented method of preparing a model for anomaly detection, wherein the model is configured to detect biological, health and ill-health traits and signatures associated with the anomaly in complete blood count (CBC) data, the method comprising: receiving CBC data from one or more data sources, wherein the CBC data comprise raw and rich data generated by one or more CBC instruments; encoding CBC data using one or more machine-learning algorithms; training a classifier for biological, health and ill-health traits and signatures based on the encoded CBC data, wherein said traits and signatures comprise at least one phenotype associated with health and ill-health; and providing the model comprising the trained classifier.

In a second aspect, the present disclosure provides a method or computer-implemented method of applying a machine-learning model to detect anomaly or anomalies in an individual-based or a population-based complete blood counts (CBC) data, the method comprising: receiving the machine-learning model trained on the CBC data, wherein the machine-learning model is prepared according to the first aspect; applying the trained model to unclassified CBC data of one or more individuals; detecting the anomaly in the unclassified CBC data based on one or more biological traits; and outputting the anomaly for clinical assessment.

In a third aspect, the present disclosure provides a platform for deploying the model prepared according to the first aspect, wherein the platform comprises one or more hardware devices configured to: receive complete blood counts (CBC) data, wherein the CBC data comprise raw and rich data; standardise the CBC data based on input settings of the machine-learning model; apply the machine-learning model to the normalized CBC data; provide a classification from the model based on a configuration of the machine learning model, wherein the configuration is associated with one or more biological, health and ill-health traits and signatures; and apply the classification to detect anomaly in the complete blood counts (CBC) data for one or more individuals or populations.

In a fourth aspect, the present disclosure provides a system for applying a machine-learning model prepared according to the first aspect, wherein the system is further configured to: receive standardised CBC data; apply the machine-learning model to the normalized CBC data; provide a classification from the model based on a configuration of the machine learning model, wherein the configuration is associated with one or more biological, health and ill-health traits and signatures; and apply the classification to detect anomaly in the blood counts (CBC) data for one or more individuals or populations.

It is understood that the model provided in any of the aspects described herein may be applied to detect anomalies in blood count (CB) results from one individual or more individuals or a population for one or more traits or biological traits described herein. For example, the model deployed with a software platform may apply to the prognosis of renal cell cancer, determining various pregnancy stages, and identifying critical biomarkers in the onset of stroke or other cardiovascular diseases.

It is further understood that the methods or method steps described herein may be performed by software in machine-readable form on a tangible storage medium e.g. in the form of a computer program comprising computer program code means adapted to perform all the steps of any of the methods described herein when the program is run on a computer and where the computer program may be embodied on a computer-readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The options or optional features described in any of the following sections may be combined as appropriate, as would be apparent to a skilled person, with any one or more aspects of the invention.

### BRIEF DESCRIPTION OF THE DRAWINGS

Embodiments of the invention will be described, by way of example, with reference to the following drawings, in which:

FIG. 1 is a flow diagram illustrating an example of model preparation for use in anomaly detection according to the invention;

FIG. 2 is a pictorial diagram illustrating an example of CBC test workflow according to the invention;

FIG. 3 is a pictorial diagram illustrating high-dimensional feature space of the model according to the invention;

FIG. 4 is a pictorial diagram illustrating an example of the high-dimensional input feature space according to the invention;

FIG. 5 is a pictorial diagram illustrating an example of results from a trained classifier according to the invention;

FIG. 6 is a pictorial diagram illustrating an example of autoencoder data compressed via autoencoder into low-dimensional feature space which has been represented in 2D according to the invention;

FIG. 7 is a pictorial diagram illustrating an example of interpretable results associated with model features that correspond with features in the dataset according to the invention;

FIG. 8 is a pictorial diagram illustrating the importance of various features of the CBC test used by the model according to the invention; and

FIG. 9 is a pictorial diagram illustrating an example of aggregate reconstruction error generated by the model with respect to the renal cell caner according to the invention.

### DETAILED DESCRIPTION

Complete Blood Counts or Full Blood Counts in other territories, CBC's hereafter, are one of the world's most common clinical tests, with approximately 3.6 billion (bn) being performed each year worldwide. They are critical to decision making by clinical care team members and inform the taking of clinical interventions in nearly all settings of health care delivery, community or primary care, secondary care at typical normal hospitals, advanced care in tertiary referral hospitals providing advanced care). However, in current practice only a limited number of the summary level measurements are considered manually on a patient-by-patient basis to reach a decision about health versus ill-health and summary level measurement results are interpreted against normal sex-stratified population ranges defined by the mean of the results in a given population of males or females, plus or minus 2.5×standard deviation. Specific normal ranges are defined for new-borns and minors with an age in years in the first decade. Doctors and scientists skilled in the art of normal blood physiology and blood diseases will use additional summary results to inform or exclude a more precise diagnosis. However all together a limited number of measurement results are used in common and advanced medical practice and additional “high level” and all of the raw measurement data being unused, unconsidered, and generally discarded as data is overwritten.

There are many variations of the CBC test however the basic test principles are the same. During the test, a sample of blood is taken and analysed using an automated haematology analyser instrument. Inside the automated instrument a small volume of a blood sample is mixed with specific dyes and reagents, the cells are then suspended in a flow stream and one by one pass through several different detectors/measurement devices, in a similar fashion to flow-cytometry. Several different types of measurement devices are used, examples include: 1) Lasers—in which the light refraction/scatter/absorbance patterns resulting from the stained cell passing through different angled laser beams is measured and 2) Electrical impedance using the Coulter principle—cells are suspended in a fluid carrying an electrical current, and as they pass through a small opening (an aperture), they cause decreases in current because of their poor electrical conductivity. The amplitude of the voltage pulse generated as a cell crosses the aperture correlates with the amount of fluid displaced by the cell, and thus the cell's volume, while the total number of pulses correlates with the number of cells in the sample.

Various calculations are then performed using these “raw” measurements to produce “high level” summary statistics such as Red Blood Cell-, White Cell- and Platelet-count, and haemoglobin concentration which are then reported. White cells are differentiated based on the measurements in the five different types, three being the granulated polymorphonuclear cells or granulocytes, named neutrophils, eosinophils and basophils and the two remaining ones being mononuclear cells, names lymphocytes and monocytes. Members of the clinical care team compare a limited number of these “high level” values to standardised population reference ranges and inform their diagnosis. As mentioned above the high-level CBC results are used in a broad way to inform or exclude diagnosis for a wide range of pathologies and illnesses such as anaemia (low level of haemoglobin), thrombocytopenia or thrombocytosis (number of platelets below and above the population normal range thresholds), leukocytopenia and leukocytosis (number of white blood cells below and above the population normal range thresholds). A high number of white blood cells combined or not combined with anaemia and/or thrombocytopenia is also a ‘warning signal’ for a possible leukaemia diagnosis. All together the routine CBC is a sensitive test to detect states of ill-health but the test result is not specific. CBC results are also used in maternity care and more broadly in population health screening programmes as a normal result excludes many pathologies. Currently no automated machine learning-based analysis methods are routinely applied to CBC data to inform diagnoses or prognosis. The use of CBC data as indicators for potential biomarkers or indications for human diseases, disease responses, conditions, states, or treatment responses is untapped in field.

Data sources include but are not limited to Rich CBC data-processed summary statistics directly output by CBC instruments such as haematology analyzers which also includes all previously described ‘high-level’ measurements; Raw CBC laser measurement data-raw measurement data from the CBC machines, including chemical staining, electrical, and laser; where CBC data sources may be from any sample source, including Primary, Secondary and Tertiary Hospital Care. Data also include measurement results on samples taken for population health screening programmes, maternity-care screening programmes, screening programmes applied to donors of blood, platelets or plasma, cohort population studies for research and other sample collection, such as but not limited to CBC tests for life insurance, other insurances, clinical studies and trials performed for obtaining regulatory approvals for new drugs, devices and vaccines.

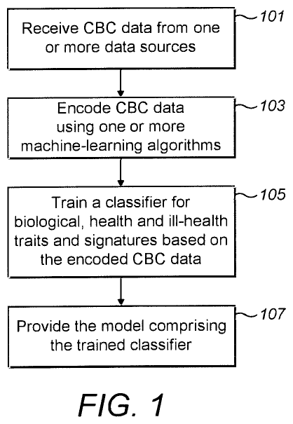

It is understood that the examples and results provided below in accordance with the above and any advantages associated with the invention can be understood by the skilled person in relation to FIGS. 1 to 9 and the studies described in the Appendix.

The example methods include 1) compression of human or animal CBC counts data from any device to obtain a low dimensional representation of the data through use of our machine-learning algorithms (e.g. Autoencoder or Variational Autoencoder); 2) classification of traits, including clinically informative disease phenotypes, in an individual using the compressed data using machine-learning methods (e.g. XGBoost, Random Forest); 3) disease detection via anomaly at the individual (e.g. individual is unwell, has anaemia, or acute viral infection) and population (e.g. a disease outbreak event has occurred in Cambridgeshire) level using the compression and classification algorithms described above; 4) algorithms and software platform for ingestion of the rich CBC results and harmonisation of results-including local analysis using on-board PCIE devices, computers, or clusters AND a cloud based analysis platform.

More specifically, in this example method, the compression step reduces model complexity and avoids over-fitting the CBC data. The compression may be accomplished using an autoencoder. The autoencoder works by training a pair of neural networks, an encoder and a decoder. The encoder compresses the input data into a lower dimension. The CBC data is encoded into N features. The decoder takes those N features as input and then reconstructs the original data. In one example, a feature space that comprises 86 features is reduced to a smaller 8-dimensional latent space. The latent space comprises the information of the 86-dimensional CBC data. The smaller compressed space may be seen as a surrogate of the higher dimensional data.

Both autoencoder and decoder networks are trained by penalizing any reconstruction differences between the input data and the reconstructed data and update the weights in the neural network to ensure that the reconstruction is as accurate as possible. The autoencoder may also be trained to encode a particular distribution of the CBC data.

In order to correct for the deviation in samples due to machine, time of day, month of the year, time between sample draw and analysis, and improve on scalability and reduce the computational complexity. The autoencoder in the model architecture may be further improved by removing the dependency on a prediction task. This allows the compressed representation to generalise to other tasks, not simply the one task it has been trained for and ensures the latent representation remains true to the original data, ensuring a form of regularisation. This approach scales to many domains, as we simply add further terms to the loss function, and to many elements within each domain as the domain classifier head is simply a multi-layer perceptron with an equal number of output neurons to those elements in each domain.

The above method is implemented by using one or more standardisation techniques. These techniques include improvements over current shortcut learning prevention techniques based on feature disentanglement in which a task specific classifier and domain specific classifier are used to force models to learn features relevant to the classification problem rather than features relevant to domain specific biases in the input data. Specifically, our method is novel and improved over other methods as the task specific classifier component of the model is replaced by minimisation of autoencoder based reconstruction error. This modification removes the dependency on a specific prediction task which current models have yielding two major benefits: 1) The resulting latent data representation output by the model can be used for other generalised downstream analysis rather than just to make a specific classification, and 2) The resulting latent data representation remains true to the original data, ensuring a form of regularisation. The improved downstream results of our pre-processing method for the implementation are detailed in Appendix Section IV. Standardisation between machines as well as in accordance with Table 2.

Following the compression step, a portion of the encoded CBC data is used to train a classifier. The classifier may be XGBoost, Random-Forest, Logistic Regression, a combination of classification models, or the most appropriate model for the classification problem at hand. In one example, 80% of the encoded data is used to train a classifier to classify the donors as male or female based on their CBC data. Five-fold cross-validation is used for this training. The 20% remaining data (unseen to the model) is used for validation based on model sensitivity and specificity. In classifying donor gender, there are latent features that help determine whether the patient is male or female. At least one latent feature is shown to correspond to the features in the data.

It is understood that the model implemented above and trained using the data described above may be used for applications as exemplified in the Appendix. These applications may involve the use of different data or data derived from different sources. Such data may be associated with and exhibiting one or more biological traits described herein.

A biological trait that may be selected from any one or more of diseases, disease responses, conditions, states, or treatment responses such as: 1) Bacterial, Viral (known ones and new unknown ones), or Parasitic infection, 2) Cancers, particularly cancers of the blood stem cells and its progeny, but also solid organ cancers at multiple stages using CBC data and above methods; 3) Cardiovascular diseases, particularly states of advanced atherosclerosis, angina pectoris, acute coronary syndrome, ST-segment elevated myocardial infarction and thrombotic stroke; 4) Metabolic disorders, like Type I (insulin-dependent), Type II diabetes, other endocrinological disorders (e.g. hypothyroidism, hyperthyroidism), metabolic disorders causal of, or accompanying obesity; 5) Autoimmune and allergic diseases, and particularly exacerbations of autoimmune diseases, as illustrated by e.g. inflammatory bowel disorders (Crohn's Disease and Ulcerative Colitis), rheumatoid arthritis, systemic lupus erythematosus, multiple sclerosis lupus, autoimmune thrombocytopenia; and allergies, including hay-fever, house dust mite, food allergies, 6) Mental ill-health, particularly mental ill-health causally linked to chronic inflammatory states; 7) Rare inherited diseases of the blood stem cell and its progeny, and also rare diseases of other organ systems where the function-modified gene which is causal for the rare disease is transcribed in the blood stem cell or its progeny; 8) Response to drug treatment/administration, including detection of signatures of commonly occurring side effects of drugs; 8) Prediction of disease progression, exacerbations, relapse and remission, particularly but not uniquely for autoimmune diseases and inflammatory disorders; 9) Identification of groups of individuals with specific target phenotype who may benefit from a certain medical intervention versus individuals who may be harmed by the same intervention (e.g. individuals at risk of cardiovascular disease in whom platelets have or have not been effectively inhibited by dual or triple anti-platelet drug therapy with aspirin, ADP-receptor inhibitors and fibrinogen-receptor inhibitors); 10) Health and ill-health in relation to pregnancy or the stages of pregnancy (i.e. characteristics exhibit during pregnancy).

It is understood the model described herein may be suitable for any one or more of the above-selected traits. The model may be applied and trained using appropriate training data with respect to each of the traits in order to provide such results as provided in the Appendix. The results of the model are applicable to assess or make predictions for a condition associated with health such as pregnancy or ill-health such as cancer, a metabolic disease, a cardiovascular disease, an autoimmune disease or allergy, a mental health disorder, a rare inherited disease, and a condition found in community care or secondary and tertiary hospital care.

In one example, the biological trait may be a type of cancer, more specifically Renal Cell Carcinoma that is known to affect 13,000 people each year in the United Kingdom and has a 50% 5-year survival rate. Practically, this means that 36 people in the UK will be diagnosed with RCC each day-half of whom will die within 5 years. Early detection of RCC is key in achieving optimal treatment outcomes, however diagnosis of RCC remains extremely difficult with the classical diagnostic symptoms of haematuria, pain and abdominal mass now recognised as being rare—and other symptoms, if present at all, can be vague, non-specific and delayed in onset. Due to the insidious nature of the disease over 60% of RCC cases are discovered incidentally when disease is at an advanced stage. Further details on the study are provided in Appendix Section III. Renal Cell Carcinoma Case Study.

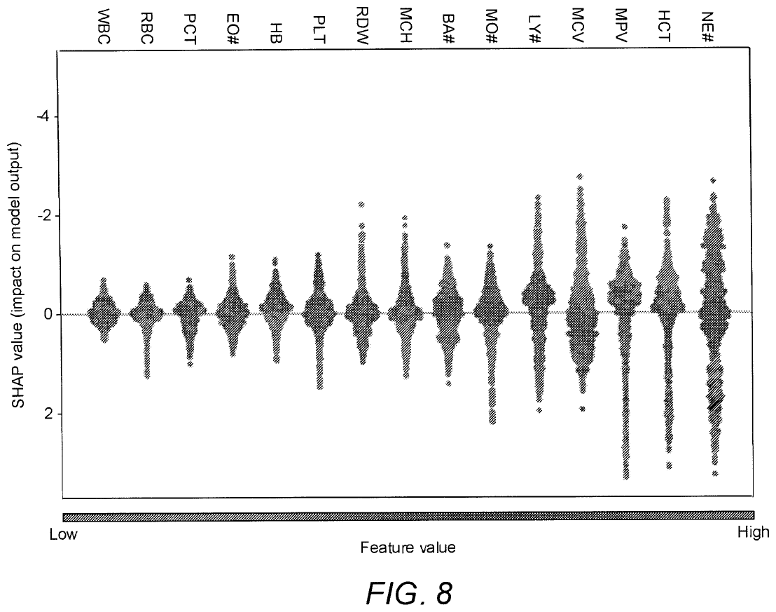

It is understood that the data generated from the study is used to train the model herein described. The results may be applicable for assessing whether a patient is likely to be suffering from RCC given analysis of their CBC test data using the model. The results may be used for prognosis or diagnostic purposes, for example, referring a patient for further investigation as it provides some decision support on whether the individual has RCC or otherwise. Several important CBC test features that are different between RCC patients and the average GP patients such as: Neutrophil Count (NE #), HCT (Haematocrit), MPV (Mean Platelet Volume) are identified as a result according to FIG. 8. The identification of these features provides improved ways of how disease progression may be assessed in respect of detecting RCC using CBC data, based on the methods described herein.

In another example, the biological trait may be a cardiovascular disease, i.e. strokes and heart attacks. A study comprises 5,036 patients who experienced a stroke and were admitted to CUH with a CBC recorded within a day of admission. Further details of the study are provided in Appendix Section I. Cardiovascular studies. As part of the study, various blood biomarkers are identified by applying the model herein described. The identified blood biomarkers correspond to each of the cohorts suffering from cardiovascular disease. In particular, there are statistically significant differences in the blood biomarker, neutrophil counts, as shown according to Appendix Section I. Chart A. It is understood that the model trained on appropriate data described herein may be used to identify risk groups, diagnose and predict outcomes for cardiovascular disease.

In yet another example, the biological trait may be characteristics exhibited during stages of pregnancy or at one point during pregnancy. The model is trained using data collected from women who have CBC in the interval. Detail of the study and the data used for training is further described in Appendix Section II. Pregnancy studies. Applying the model allows identification of significant features. These features separate the stages of pregnancy. In particular, the significant features are: (a) Total peroxide; (b) WBC from peroxidase method; and (c) Mode Lymphocyte count. This is further described in Appendix Section II. Chart A. Other significant features in relation to cells and cellular components, in particular platelets, neutrophils, haemoglobin, while blood cells, lymphocytes, are provided according to Appendix Section II. Chart B. The identification of these significant features or biomarkers using the model described herein provides a means to evaluate and for the early detection of complications during pregnancy, including preeclampsia and pregnancy-induced diabetes.

In yet another example, the biological trait may be characteristics exhibited in relation to metabolism, for example, obesity or the prediction thereof. It is understood that there may be biomarkers in CBC data indicating different levels of obesity as defined by the Body Mass Index (BMI), and these biomarkers may be identified by the model for obesity prediction. In an experiment, CBC data from the INTERVAL blood donor may be used and as input for model. The dataset is divided into 5 weight classes for different levels of obesity as defined by NHS England. These are as follows: underweight (BMI [symbol] 18.5), healthy (BMI: 18.5-24.9), overweight (BMI 25.0-29.9), obese (BMI 30.0-39.9) and severely obese (BMI 40.0+). In addition, the CBC data may be used to identify the sex of an individual and it is well known that there are biological weight differences between males and females; therefore, analysis is carried out for male and female blood donors separately to avoid sex related bias. The following table shows the number of CBC tests available for donors in each weight class.

TABLE 1

| | | |
|---|---|---|
| Obesity Class | Male | Female |

| | | | | |
|---|---|---|---|---|
| Underweight | 58 | (0.6%) | 98 | (0.9%) |
| Healthy | 3759 | (37.2%) | 5203 | (49.1%) |
| Overweight | 4415 | (43.7%) | 3098 | (29.2%) |
| Obese | 1757 | (17.4%) | 1918 | (18.1%) |
| Severely Obese | 121 | (1.2%) | 279 | (2.6%) |

| | | |
|---|---|---|
| Total | 10110 | 10596 |

Using only uncorrelated ‘high level’ CBC features in the dataset, weight class of a donor are classified based only on their CBC data. The data was split into a development (⅔ of the data) and holdout (⅓ of the data) sets. The model was trained using 5-fold cross-validation. For the female cohort, the mean validation AUC is 0.830667 and internal holdout sensitivity is 0.770886 and specificity is 0.737313. For the male cohort, the mean validation AUC is 0.829957 and internal holdout sensitivity is 0.734328 and specificity is 0.775949. A caveat of this analysis is that there are very few samples from underweight and severely obese blood donors due to the selection biases for blood donation.

Further, herein described method may include: 1) Detection of known or novel pathogen outbreaks in a population (e.g. pathogen agnostic detection of SARS-CoV-2 infection outbreak in Cambridgeshire); 2) The model may be configured to capture temporal dependencies in the data; The above where the model is configured to interpret CBC results from population where multi-pathogen infection is endemic (e.g. in low and middle income countries). It can be appreciated that the temporal dependencies in the data or the change over time in the patient CBC may be an important indicator with respect to, for example, the prognosis of renal cell cancer and to make assessments during pregnancy. Applying the indicator would effectively increase the accuracy of the model results.

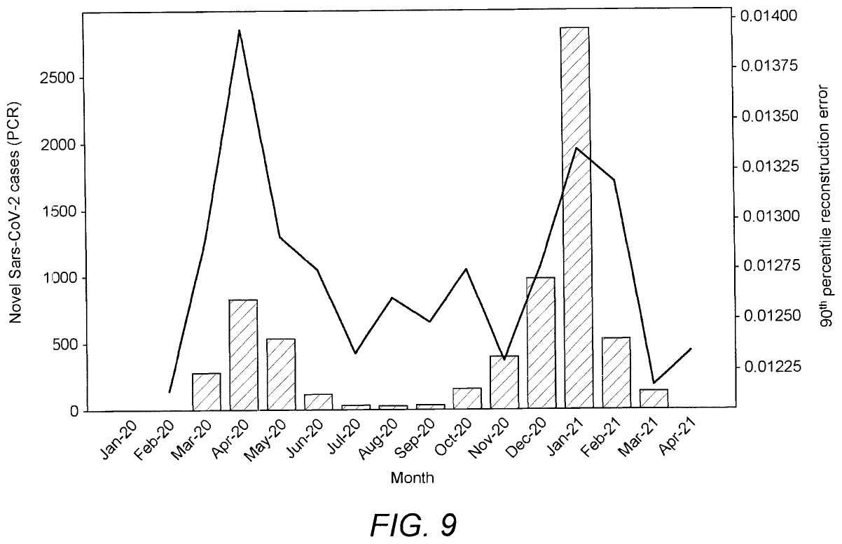

In another example, data from all of the complete blood counts performed during a time period may be encoded and processed, i.e. from Addenbrooke's lab data in 2019, to get a representation of the patient distribution for that time period. Further data from a later period (i.e. 2020 and 2021) may be incorporated into the model. By comparing model error for time-dependent CBC samples, pandemic events such as COVID-19 in a region may be identified, allowing for scalable and cheap population screening methods for pathogen outbreaks or other anomaly detection. More specifically, pathogen outbreak events such as COVID-19 may be identified and forecasted by the model to the extent of interpreting CBC results from a population. An example of this is shown according to FIG. 9 and described in the following sections.

In relation to the above example, the model may comprise an autoencoder that is trained using data from 103,219 R-CBC measurements performed on the Cambridgeshire population between October 2019 and January 2020 when no SARS-CoV-2 cases were expected. The model was then used to compress and reconstruct the remaining 404,215 R-CBC measurements performed between February 2020 and April 2021. Model is proposed to make errors as shown in FIG. 9 as it encounters R-CBC measurements it had not been trained with before (i.e. those from COVID-19 patients).

The above where the method includes: 1) Ingestion of rich and raw CBC measurement data using automated software and analysis pipeline; 2) Standardisation of data from CBC instruments from different manufacturers; 3) Automated detection of deviation by an instrument of measuring CBC parameters accurately.

Following the above for Rich data: 1) Data compression using self-/semi-/un-/supervised methods; 2) Classification of data in the compressed space using self-/semi-/un-/supervised.

Following the above for Raw data: 1) Clustering of raw data using deep neural network techniques or computer vision techniques; 2) Feature engineering from the clustering output; 3) From now, as above for Rich data.

Following initial analysis: 1) Aggregation of analysed data from all sources; 2) Training of self-/semi-/un-/supervised methods for detection of anomalies in population samples

The above method may include 1) Interpretability techniques for analysis of learned features and latent space; Algorithms for active learning/model hyperparameter tuning based on output results.

An at scale analysis platform: 1) Streaming CBC data from testing locations to central analysis compute environment; 2) Local analysis of CBC data and streaming of analysis results to central compute environment in a federated learning style approach; 3) Analysis of collated data for population health monitoring and disease outbreak detections.

### Example Applications of Model Results

In relation to the above, multiple semi-supervised and unsupervised models have been developed, which can be used to analyse the “rich” and “raw” CBC data and detect various important clinical events e.g.

1) We can use the rich and raw data to infer Sex (Male or Female) with 0.95 Area Under Curve (AUC) internal validation data and 0.87 sensitivity and 0.89 Specificity on the internal holdout set. In an external blood donor dataset called STRIDES we have 0.85 sensitivity and 0.85 specificity and for another blood donors dataset named COMPARE 0.87 sensitivity and 0.80 specificity. 2) Obesity-internal validation-0.81 AUC for internal holdout 0.73 sensitivity, 0.70 specificity. 3) Hospital versus community samples (non hospital)-internal validation 0.88 AUC, holdout 0.80 sensitivity and specificity. 4) Aggregation of the data allows us to perform other population wide analyses such as identification of outbreaks of infectious diseases. We have done this for infection with SARS-CoV2 in samples from the wider Cambridgeshire population, with detection of infection in samples of venous blood obtained from individuals attending community-based General Practitioner (GP) clinics or patients seen in outpatients and inpatients at Cambridge University Hospitals.

### Exemplary Model Implementations

TABLE 2

| | Renal cell | | |
|---|---|---|---|
| Study | carcinoma | Cardiovascular | Pregnancy |
| Input data formats | Rich and Raw data | Rich and Raw data | Rich and Raw data |
| | from automated | from automated | from automated |
| | Complete Blood | Complete Blood | Complete Blood |
| | Count (CBC) | Count (CBC) | Count (CBC) |
| | analysers. | analysers. | analysers. |
| Network topology | All layers fully | All layers fully | All layers fully |
| (nodes, number of | connected | connected | connected |
| layers, arrangement | L1 = CBC Input | L1 = CBC Input | L1 = CBC Input |
| of nodes and layers | L2 = 64 | L2 = 64 | L2 = 64 |
| | L3 = 32 | L3 = 32 | L3 = 32 |
| | L4 = 8 - latent | L4 = 8 - latent | L4 = 8 - latent |
| | L5 = 32 | L5 = 32 | L5 = 32 |
| | L6 = 64 | L6 = 64 | L6 = 64 |
| | L7 = | L7 = | L7 = |
| | Reconstruction | Reconstruction | Reconstruction |
| Applicable Models | Autoencoders, | Autoencoders, | Autoencoders, |
| | Neural Networks, | Neural Networks, | Neural Networks, |
| | XGBoost + Logisti | XGBoost | XGBoost |
| | Regression | Classifiers | Classifiers |
| | Classifiers | | |
| Connectivity | Feed forwards | Feed forwards | Feed forwards |
| (feedback, | | | |
| feedforward) | | | |
| Recurrency | No | No | No |
| Transfer or activation | Logistic/Relu | Relu | Relu |
| functions | | | |
| Learning paradigms - | Self-supervised/ | Self-supervised/ | Self-supervised/ |
| supervised, and | supervised | supervised | supervised |
| supervised, | | | |
| reinforcement | | | |
| How learning data is | CBC data in this | CBC data in this | CBC data in this |
| selected and now | work has been | work has been | work has been |
| training set is | generated during | generated during | generated during |
| composed | routine care of GP | routine care of GP | routine care of GP |
| | and Hospital | and Hospital | and Hospital |
| | patients. Models are | patients. Models are | patients. Models are |
| | trained using | trained using | trained using |
| | uncorrelated | uncorrelated | uncorrelated |
| | features from the | features from the | features from the |
| | rich-CBC data and | rich-CBC data and | rich-CBC data and |
| | decrypted cell-level | decrypted cell-level | decrypted cell-level |
| | laser data. | laser data. | laser data. |
| Cost functions | Reconstruction | Reconstruction | Reconstruction |
| | Loss and (1- | Loss and (1- | Loss and (1- |
| | sensitivity)\hat{ }2 + | sensitivity)\hat{ }2 + | sensitivity)\hat{ }2 + |
| | (1specificity)\hat{ }2 | (1specificity)\hat{ }2 | (1specificity)\hat{ }2 |
| | minimisation | minimisation | minimisation |
| Additional | Latent space is used | Latent space is used | Latent space is used |
| factors | for classification | for classification | for classification |

The above table provides examples of the machine-learning implementation deployed with respect to the different studies described in the Appendix. The implementation may vary for other applications of the model described in the application. The implementation is applicable to various aspects and examples of the invention as described herein

FIG. 1 is a flow diagram illustrating an example of model preparation for use in anomaly detection. The model is prepared or trained using one or more machine-learning methods described herein for detecting anomalies in the complete blood count (CBC) data. In particular, the model is configured to detect biological, health and ill-health traits and signatures associated with the anomaly in CBC data.

In step 101, CBC data from one or more data sources is received. The CBC data comprise raw and rich data generated by one or more CBC instruments. In step 103, CBC data is encoded using one or more machine-learning algorithms. In step 105, a classifier is trained to classify biological, health and ill-health traits and signatures based on the encoded CBC data/The traits and signatures comprise at least one phenotype associated with health and ill-health. In step 107, the model comprising the trained classifier is provided for further applications.

These applications may include but are not limited to detecting anomaly in blood count results from one individual or more individuals, or detecting at least one anomaly at a population level. The model may be deployed with a software platform, where the software platform comprises one or more hardware devices configured to pre-process the CBC data.

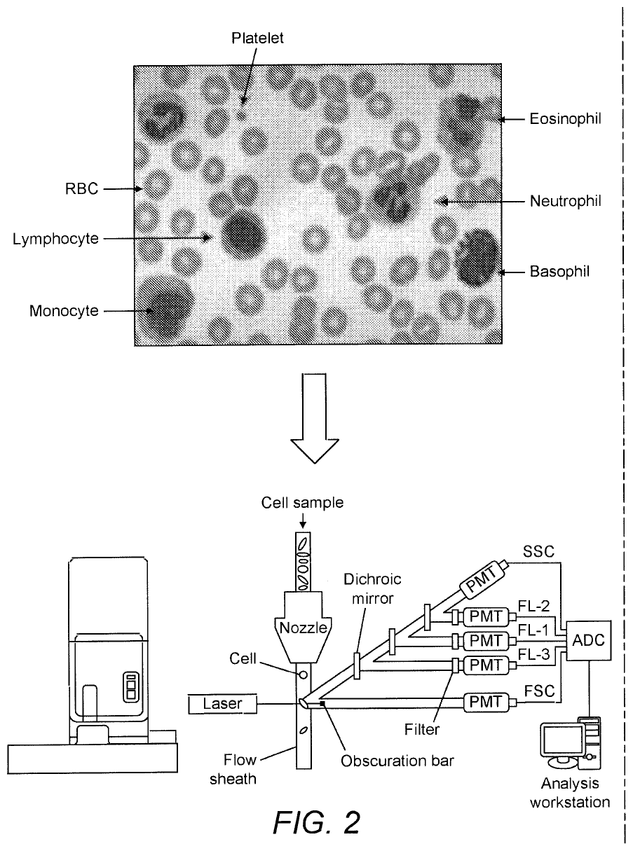

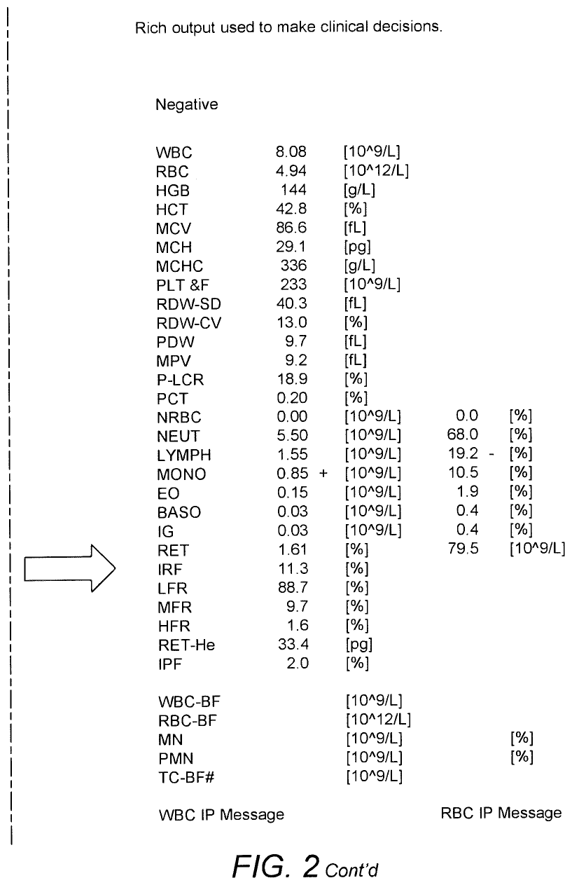

FIG. 2 is a pictorial diagram illustrating an example of CBC test workflow. The figure shows a “high level” data report generated from the model. The output report contains only a subset of the “high-level” and “rich” measurements used by the invention. In practice, a limited number of the measurements on display in the report (e.g. WBC, RBC, HGB) are presented to healthcare professionals to inform diagnoses and medical decision-making.

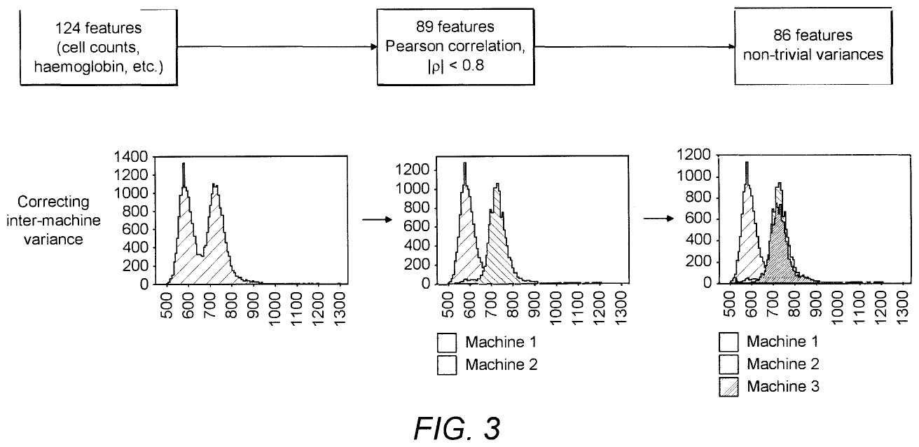

FIG. 3 is a pictorial diagram illustrating high-dimensional feature space associated with the CBC data, and standardisation of input data from different sources to account for variability.

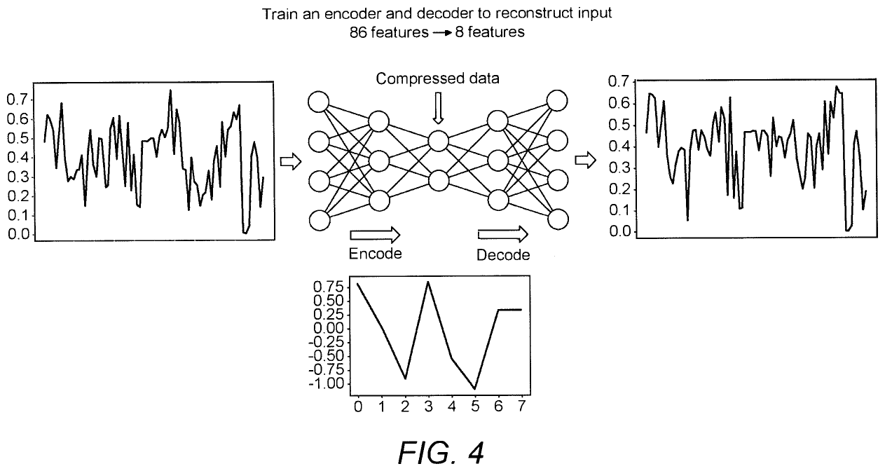

FIG. 4 is a pictorial diagram illustrating an example of the high-dimensional input feature space being compressed to—and decompressed from—a latent space using an autoencoder. Exemplary layers of the network are also shown, where the data is compressed. For example, the compressed data corresponding to the network structure where the encoder and decoder are trained to reconstruct input of 86 features to 8 features.

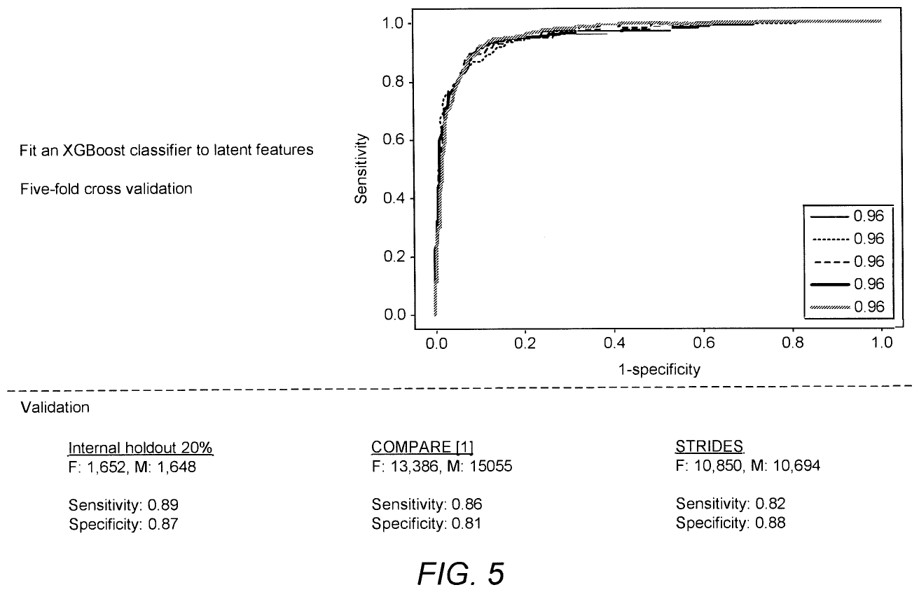

FIG. 5 is a pictorial diagram illustrating an example of results from a trained classifier that classifies traits and signatures based on the latent space encoding of CBC data.

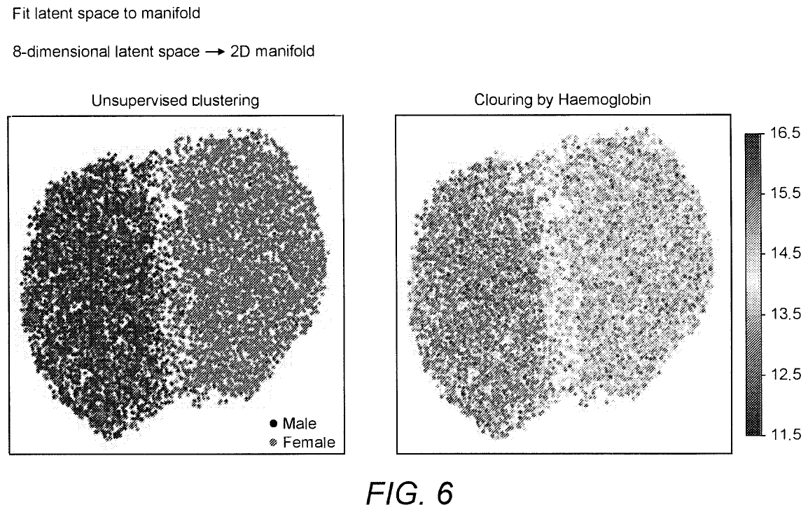

FIG. 6 is a pictorial diagram illustrating an example of autoencoder data compressed via autoencoder into low-dimensional feature space, which has been represented in 2D. The specific figures demonstrate the application of the invention in discerning Males from Females, using only CBC data and classification using features learned during autoencoder and classification model training;

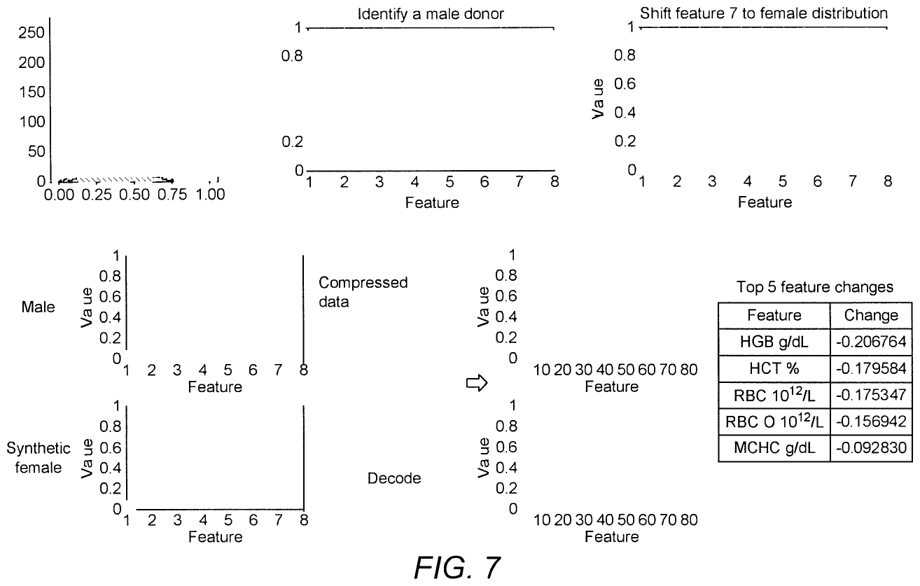

FIG. 7 is a pictorial diagram illustrating an example of interpretable results associated with model features that correspond with features in the dataset. It demonstrates the process of linking learned latent space features back to input features, compressing CBC input data for a given sample, manipulating derived features in the latent compressed space data to create an artificial encoding, reconstructing inputs from the artificial encodings using the invention, and comparing the differences observed in the artificial output data, to those observed in the original input data.

FIG. 8 is a pictorial diagram illustrating an example of RCC vs. GP CBC classification feature importance in an application for diagnosing the onset of renal cell carcinoma. Shown are in the importance of various features of the CBC test used by the model in classification of Complete Blood Counts (CBC) tests from Renal Cell Carcinoma (RCC) patients vs. those from General Practitioner (GP) patients. This is further described in the Appendix.

FIG. 9 is a pictorial diagram illustrating an example of aggregate reconstruction error over months compared to the Public Health England (at the time) PCR determined caseload in relation to the Cambridgeshire population (in Cambridge in a database). In the figure, the blue bars (X-axis 1) represent the number of new monthly cases identified by the hospital laboratory (regional test centre) using PCR. The red line (X-axis 2) represents the average 90th percentile reconstruction error generated by the model at the same time points. By setting a threshold on the Y-axis, you can trigger an outbreak investigation.

The figure shows significant increases in average monthly compression/reconstruction error rates were observed for the whole of 2020-2021 peaking during the March/April and December/January which coincides with the Cambridgeshire SARS-CoV-2 infection ‘waves’. Peak error rates correlate strongly with the number of CBC tests being performed on known SARS-CoV-2 PCR-positive individuals. This shows that we can detect the presence of these infected individuals in the population using R-CBC data. Higher error rates between June-2020 and October-2020, a period during which few new cases were identified, are explained by proportion of CBC tests being performed during this period on hospitalised COVID-19+ patients.

The above FIGS. 1 to 9 correspond to the following aspects. One aspect is a method or computer-implemented method of preparing a model for anomaly detection, wherein the model is configured to detect biological, health and ill-health traits and signatures associated with the anomaly in complete blood count (CBC) data, the method comprising: receiving CBC data from one or more data sources, wherein the CBC data comprise raw and rich data generated by one or more CBC instruments; encoding CBC data using one or more machine-learning algorithms; training a classifier for biological, health and ill-health traits and signatures based on the encoded CBC data, wherein said traits and signatures comprise at least one phenotype associated with health and ill-health; and providing the model comprising the trained classifier.

Another aspect is a method or computer-implemented method of preparing a model for detecting renal cell cancer, determining stages in pregnancy, or predicting whether a cardiovascular event will occur, wherein the model is configured to detect related biological, health and ill-health traits and signatures associated with the anomaly in complete blood count (CBC) data from a patient, the method comprising: receiving CBC data from one or more data sources, wherein the CBC data comprise raw and rich data generated by one or more CBC instruments; encoding CBC data using one or more machine-learning algorithms; training a classifier for biological, health and ill-health traits and signatures based on the encoded CBC data, wherein said traits and signatures comprise at least one phenotype associated with health and ill-health; and providing the model comprising the trained classifier, wherein the classifier is configured determine whether a patient exhibits renal cell cancer, identifying a stage in pregnancy, or predict the cardiovascular event with respect to the biomarkers learned by the model.

Another aspect is a method or computer-implemented method of applying a machine-learning model to detect anomaly in an individual-based or a population-based complete blood counts (CBC) data, the method comprising: receiving the machine-learning model trained on the CBC data, wherein the machine-learning model is prepared according to the first aspect and/or according to the option(s) described herein; applying the trained model to unclassified CBC data of one or more individuals; detecting the anomaly in the unclassified CBC data based on one or more biological traits; and outputting the anomaly for clinical assessment.

Another aspect is a platform for deploying the model prepared according to the first aspect and/or according to the option(s) described herein, wherein the platform comprises one or more hardware devices configured to: receive complete blood counts (CBC) data, wherein the CBC data comprise raw and rich data; standardise the CBC data based on input settings of the machine-learning model; apply the machine-learning model to the normalized CBC data; provide a classification from the model based on a configuration of the machine learning model, wherein the configuration is associated with one or more biological, health and ill-health traits and signatures; and apply the classification to detect anomaly in the complete blood counts (CBC) data for one or more individuals or populations.

Another aspect is a system for applying a machine-learning model prepared according to the first aspect and/or according to the option(s) described herein, wherein the system is further configured to: receive standardised CBC data; apply the machine-learning model to the normalized CBC data; provide a classification from the model based on a configuration of the machine learning model, wherein the configuration is associated with one or more biological, health and ill-health traits and signatures; and apply the classification to detect anomaly in the blood counts (CBC) data for one or more individuals or populations.

As an option, the biological traits or traits may be associated with characteristics of a cellular component or cell type. As another option, the characteristics comprise counts or quantified measurement of the characteristics. As yet another option, the characteristics comprise one or more of total peroxide quantify, white blood cell count, lymphocyte count, platelets count, neutrophil count, haemoglobin count, and lymphocytes count.

As an option, further comprising: normalizing the received CDC data before encoding. As another option, wherein said normalization comprises one or more methods configured to correct for the sample deviation due to applying the said model on two or more hardware devices. As another option, said normalization is performed applying one or more data standardisation techniques. As another option, said traits are associated with ill-health, or the presence of an infectious agent or pathogen. As another option, the traits are biological traits associated one or more cell types or cellular components. As another option, said traits correspond to an ill-health response associated with at least one state of ill-health to health or at least one state of health to ill-health, wherein said at least one state comprises onset, exacerbation, relapse, and remission. As another option, the ill-health is a condition as results of a cancer, a metabolic disease, a cardiovascular disease, an autoimmune disease or allergy, a mental-health disorder, a rare inherited disease, or is a condition found in community care or secondary and tertiary hospital care. As another option, the condition is one or more of a cancer, a metabolic disease, a cardiovascular disease, an autoimmune disease or allergy, a mental-health disorder, a rare inherited disease, or is a condition found in community care or secondary and tertiary hospital care. As another option, the cancer comprises renal cell carcinoma. As another option, the cardiovascular disease comprises stroke and heart attack. As another option, the ill-health is related to a health trait. As another option, the health traits is associated with pregnancy. As another option, the ill-health is a type of complication induced by or occurs during pregnancy. As another option, said at least one phenotype correspond to a clinically informative response based on a treatment of a drug or drug candidate, or based on a change to diet or physical activity. As another option, the treatment comprises a dosage regimen of the drug or drug candidate. As another option, the anomaly is associated with a pathogen outbreak in a population. As another option, the anomaly is associated with the presence of toxic substance to which a population has been exposed. As another option, the anomaly is associated with the presence of radiation toxicity to which a population has been exposed. As another option, the model is configured to capture temporal dependencies in the CBC data.

The above description discusses embodiments and aspects of the invention with reference to a single user for clarity. It will be understood that in practice the system may be shared by a plurality of users, and possibly by a very large number of users simultaneously.

The embodiments and aspects described above may be configured to be semi-automatic and/or are configured to be fully automatic. In some examples a user or operator of the querying system(s)/process(es)/method(s) may manually instruct some steps of the process(es)/method(es) to be carried out.

The described embodiments and aspects of the invention a system, process(es), method(s) and the like according to the invention and/or as herein described may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium or non-transitory computer-readable medium. Computer-readable media may include, for example, computer-readable storage media. Computer-readable storage media may include volatile or non-volatile, removable or non-removable media implemented in any method or technology for storage of information such as computer-readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realize that such processing capabilities are incorporated into many different devices and therefore the term ‘computer’ includes PCs, servers, IoT devices, mobile telephones, personal digital assistants and many other devices.

Those skilled in the art will realize that storage devices utilized to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realize that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments and aspects are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary”, “example” or “embodiment” is intended to mean “serving as an illustration or example of something”. Further, to the extent that the term “includes” is used in either the detailed description or the claims, such a term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, subroutines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claim.

### APPENDIX

### I. Cardiovascular Case Studies

We believe there are blood biomarkers that can be used to identify risk groups, diagnose and predict outcomes for cardiovascular diseases, including strokes and heart attacks. These populations are important, as unlike other cohorts, the CBC is performed very shortly after the incident as the person is taken to hospital quickly.

There are 5,036 patients who have experienced a stroke and been admitted to CUH with a CBC recorded within a day of admission. Initially, we focus on predicting whether a patient is likely to die within a given window from the CBC. There are 292 patients who die within 3 days, 443 within 7, 602 within 14, 698 within 21, 765 within 28, 913 within 60 and 976 within 90.

We have considered the blood biomarkers for each of these cohorts and notice statistically significant differences in the neutrophil counts. In the Figure below, we show how the neutrophil count is higher in all groups that die within 90 days, but is most elevated in the group who die within 3 days and then the elevation decays.

This suggests that we may be able to use neutrophil count, along with more detailed rich CBC data to train models to predict likely outcomes for the stroke patients. This analysis naturally extends to heart attacks and other cardiovascular diseases.

### II. Pregnancy Case Study

We use the following data in our pregnancy study for women who have a complete blood count in the interval; 348 women in early stage (10 weeks-14 weeks), 450 mid pregnancy (26 weeks-30 weeks) and 242 late stage (>=38 weeks). If there are multiple CBC results, we take the latest one. We drop all correlated features in the dataset and fit a machine learning model, using five-fold cross-validation, to a development dataset representing ⅔ of the data, with the remaining ⅓ used as a holdout set for testing.

For identification of early vs. mid pregnancy, we have an average validation AUC of 0.73 along with holdout sensitivity of 0.63 with specificity of 0.76. For early vs. late pregnancy we have an average validation AUC of 0.76 along with holdout sensitivity of 0.60 with specificity of 0.70. Finally, for mid stage vs. late stage, we have an average validation AUC of 0.70 along with holdout sensitivity of 0.70 with specificity of 0.66. These models allow us to identify significant features for the models which separate the stages of pregnancy. In particular, the following three features.

We also find statistically significant differences in several blood parameters through the course of a pregnancy, when compared to the age matched blood donors from the INTERVAL and COMPARE studies.

This leads us to believe that we can predict the stage of pregnancy for women, along with identifying the biomarkers which indicate progression through it. With such variability from a donor population, we believe this technology will allow us to identify complications during pregnancy, including preeclampsia and pregnancy induced diabetes. There is such a large difference in these markers for pregnancy at all stages, compared to the donor population, that we believe this may also be used as a flag for incidentally identifying pregnancy. It is not clear yet how early the biomarkers start to shift, as we are lucky to incidentally collect this data currently.

### III. Renal Cell Carcinoma Case Study

Renal Cell Carcinoma (RCC) affects 13,000 people each year in the United Kingdom and has a 50% 5-year survival rate (https://www.cancerresearchuk.org/health-professional/cancer-statistics/statistics-by-cancer-type/kidney-cancer #heading-Zero). In real terms this means that 36 people in the UK will be diagnosed with RCC each day-half of whom will die within 5 years.

Previous studies have shown that early detection of RCC is key in achieving optimal treatment outcomes, however diagnosis of RCC remains extremely difficult with the classical diagnostic symptoms of haematuria, pain and abdominal mass now recognised as being rare—and other symptoms, if present at all, can be vague, non-specific and delayed in onset. Due to the insidious nature of the disease over 60% of RCC cases are discovered incidentally when disease is at an advanced stage (https://www.ncbi.nlm.nih.gov/pmc/articles/PMC7223292/).

Given the role of the Kidney in erythropoietin (EPO) production, a regulator of blood cell production, and previous evidence that CBC derived blood indices have a correlation with survival in RCC patients we hypothesise that Complete Blood Count (CBC) measurement data may contain valuable biological information relevant to RCC which could lead to earlier detection and diagnosis of the disease.

In the CUH EpiCov dataset we were able to identify 2,585 unique patients with a diagnosis of Renal Cell Carcinoma. Of these individuals 409 had multiple diagnoses >2 years apart suggesting relapse or disease of their other Kidney. We chose to focus on the primary episode of RCC in each patient leaving 2,176 unique patients/episodes in the dataset. In total data from 12,793 CBC's were available for these patients.

In a proof-of-principle analysis we took the First CBC test performed within a 1-year window prior to RCC diagnosis for each episode—this left 846 CBC tests in the ‘case set’ (referred to form here as RCC CBC tests). For a control set we identified 1.7 M CBC tests from patients who only visited primary care settings and who were not admitted to hospital (i.e., General Practitioner CBC tests, referred to from here as GP CBC tests). To avoid class imbalance issues GP CBC tests were randomly down sampled to a set of 1,692 to form a final ‘control set’ using a method which ensured the age and sex distributions of the source patients was similar to the patient population who provided the RCC CBC tests. In total data from 2,583 CBC tests were used. Using only uncorrelated ‘high level’ CBC features in the dataset we fit a machine learning model to classify RCC CBC vs. GP CBC, using five-fold cross-validation, to a development dataset representing ⅔ of the data, with the remaining ⅓ used as a holdout set for testing. For identification of RCC CBC vs. GP CBC we observed an average validation AUC of 0.81 and a holdout sensitivity of 0.64 and specificity of 0.75.

This analysis allowed us to identify several important CBC test features that are different between RCC patients and the average GP patients such as: Neutrophil Count (NE #), HCT (Haematocrit), MPV (Mean Platelet Volume) (see FIG. 8). These promising initial results warrant further investigation into CBC based detection of RCC as test sensitivity of 64% is a dramatic improvement of current reported symptom based RCC detection rate of 40%. Addition of the rich laser CBC measurement to the model and pre-processing (see IV. Standardisation between machines) with the full analysis methodologies described in this patent may significantly improve model performance. Furthermore, investigation of RCC CBC's falsely classified as GP CBC's by the model revealed that 62% were from the first half of the pre-diagnosis year, i.e., were taken >183 days prior to RCC diagnosis—meaning advanced RCC disease is less likely. We can use electronic healthcare record data to better assess at which disease progression stage we can detect RCC using CBC data and construct better model evaluation experiments with a focus on specific disease stages.

Iv. Standardisation Between Machines

CBC data is inherently messy due to two main root causes. Firstly, the clinical practice between the blood being taken and analysed can lead to large changes in the blood. For example, if a sample is left for a long time before being analysed the WBC count declines significantly and the temperature of storage for the sample also significantly affects the sample. Secondly, the CBC instruments themselves are highly variable depending on many factors, including the time of day, temperature of the room, the time the machine has been working for.

We have been applying several approaches to remove the bias due to the machines. In particular, we consider approaches based on the use of mathematical splines to correct for sample deviation, following the approach of (Astle, Cell 2016), to correct for the deviation in samples due to machine, time of day, month of the year, time between sample draw and analysis. However, this approach does not scale to many machines and is computationally expensive.

Therefore, we follow the approach of Robinson et al. (https://www.ncbi.nlm.nih.gov/pmc/articles/PMC7885941/) and use a machine learning method to extract features which are invariant under different domains. This has been applied previously to imaging data with a defined prediction task as an outcome. We have further developed this method, to remove the dependency on a prediction task and to incorporate an autoencoder in the model architecture. The first of these allows the compressed representation to generalise to other tasks, not simply the one task it has been trained for. The second adaptation ensures the latent representation remains true to the original data, ensuring a form of regularisation. This approach scales to many domains, as we simply add further terms to the loss function, and also to many elements within each domain, as the domain classifier head is simply a multi-layer perceptron with an equal number of output neurons to those elements in each domain.

This model has been trained using INTERVAL data, with two machines, and COMPARE for testing and for sex identification, the sensitivity of the model improved from 0.85 to 0.91 and specificity from 0.88 to 0.93. The model has also been trained using synthetic data with major boosts also observed.

Extending beyond this, we can now apply this framework for the pandemic surveillance tool to standardise samples between countries, manufacturer and machine at scale. Therefore the representation of the blood will be purely the invariant features between human blood samples, not influenced by the clinical collection and machine biases.

1. A computer-implemented method of preparing a model for anomaly detection, wherein the model is configured to detect biological, health and ill-health traits and signatures associated with the anomaly in complete blood count (CBC) data, the method comprising:
receiving CBC data from one or more data sources, wherein the CBC data comprise raw and rich data generated by one or more CBC instruments;
encoding CBC data using one or more machine-learning algorithms;
training a classifier for biological, health and ill-health traits and signatures based on the encoded CBC data, wherein said biological, health and ill-health traits and signatures comprise at least one phenotype associated with health and ill-health; and
providing the model comprising the trained classifier.

2. The method of claim 1, further comprising:
applying the model to detect anomaly in blood count (CB) results from one individual or more individuals.

3. The method of claim 1, furthering comprising:
applying the model to detect at least one anomaly at a population level.

4. The method of claim 1, further comprising:
deploying the model with a software platform, wherein the software platform comprises one or more hardware devices configured to pre-process the CBC data.

5. The method of claim 1, further comprising: normalizing the received CDC data before encoding.

6. The method of claim 5, wherein said normalization comprises one or more methods configured to correct for a sample deviation due to applying the said model on two or more hardware devices.

7. The method of claim 5, wherein said normalization is performed applying one or more data standardisation techniques.

8. The method of claim 1, wherein said traits are associated with ill-health, and/or the presence of an infectious agent or pathogen.

9. The method of claim 8, wherein the traits are biological traits associated one or more cell types or cellular components.

10. The method of claim 1, wherein said traits correspond to an ill-health response associated with at least one state of ill-health to health or at least one state of health to ill-health, wherein said at least one state comprises onset, exacerbation, relapse, and remission.

11. The method of claim 8, wherein the ill-health is a condition as results of a cancer, a metabolic disease, a cardiovascular disease, an autoimmune disease or allergy, a mental-health disorder, a rare inherited disease, or is a condition found in community care or secondary and tertiary hospital care.

12. The method of claim 11, wherein the cancer comprises renal cell carcinoma.

13. The method of claim 11, wherein the cardiovascular disease comprises stroke and heart attack.

14. The method of claim 1, wherein the ill-health is related to a health trait.

15. The method of claim 14, wherein the health traits is associated with pregnancy.

16. The method of claim 1, the ill-health is a type of complication induced by or occurs during pregnancy.

17. The method of claim 1, wherein said at least one phenotype correspond to a clinically informative response based on a treatment of a drug or drug candidate, or based on a change to diet or physical activity.

18. The method of claim 17, wherein the treatment comprises a dosage regimen of the drug or drug candidate.

19. The method of claim 1, wherein the anomaly is associated with a pathogen outbreak in a population.

20. The method of claim 1, wherein the anomaly is associated with the presence of toxic substance to which a population has been exposed.

21. The method of claim 1, wherein the anomaly is associated with the presence of radiation toxicity to which a population has been exposed.

22. The method of claim 1, wherein the model is configured to capture temporal dependencies in the CBC data.

23. A computer-implemented method of applying a machine-learning model to detect anomaly in an individual-based or a population-based complete blood counts (CBC) data, the method comprising:
receiving the machine-learning model trained on the CBC data, wherein the machine-learning model is prepared according to claim 1;
applying the trained model to unclassified CBC data of one or more individuals;
detecting the anomaly in the unclassified CBC data based on one or more biological traits; and
outputting the anomaly for clinical assessment.

24. (canceled)

25. The method of claim 23, wherein the biological traits are associated with characteristics of a cellular component or cell type.

26. The method of claim 25, wherein the characteristics comprise counts or quantified measurement of the characteristics.

27. The method of claim 25, wherein the characteristics comprise one or more of total peroxide quantify, white blood cell count, lymphocyte count, platelets count, neutrophil count, haemoglobin count, and lymphocytes count.

28. A platform for deploying a machine-learning model prepared according to claim 1, wherein the platform comprises one or more hardware devices configured to:
receive complete blood counts (CBC) data, wherein the CBC data comprise raw and rich data;
standardize the CBC data based on input settings of the machine-learning model to generate normalized CBC data;
apply the machine-learning model to the normalized CBC data;
provide a classification from the model based on a configuration of the machine learning model, wherein the configuration is associated with one or more biological, health and ill-health traits and signatures; and
apply the classification to detect anomaly in the complete blood counts (CBC) data for one or more individuals or populations.

29. (canceled)

30. A system for applying a machine-learning model prepared according method claim 1, wherein the system is further configured to:
receive standardized CBC data;
apply the machine-learning model to the standardized CBC data;
provide a classification from the model based on a configuration of the machine learning model, wherein the configuration is associated with one or more biological, health and ill-health traits and signatures; and
apply the classification to detect anomaly in the blood counts (CBC) data for one or more individuals or populations.
